(19) United States
(12) Patent Application Publication
(10) Pub. No.: US 2006/0039996 A1
(43) Pub. Date: Feb. 23, 2006

(54) Method for treating sepsis

(76) Inventor: Craig R. Palmer, San Francisco, CA (US)

Correspondence Address: MAYER, BROWN, ROWE & MAW LLP, P.O. BOX 2828, CHICAGO, IL, 60690-2828, US

(21) Appl. No.: 11/083,148

(22) Filed: Mar. 17, 2005

Related U.S. Application Data
(60) Provisional application No. 60/553,736, filed on Mar. 17, 2004.

(51) Int. Cl. A61K 33/14; A61K 33/40

(52) U.S. Cl. 424/661; 424/616

(57) ABSTRACT

Compositions and methods for treating or preventing sepsis in an animal are disclosed. The method comprises administering an electrolyzed saline solution to the animal. In one embodiment, the electrolyzed saline solution comprises ozone and at least one active chlorine species. The electrolyzed saline can further comprise at least one active hydrogen species.

### BACKGROUND

Sepsis is a systemic inflammatory response to infection. Sepsis is one of the most important causes of adult respiratory diseases and lung injury; however, the critical interplay between infection and the systemic response leading to lung injury is poorly understood. Furthermore, sepsis is a serious animal health risk factor. Sepsis is an animal health risk factor in at least two regards. First, any bacterial infection, be it in the lungs, skin, bowel, urinary tract, bone or vital organs, can lead to sepsis, causing vital organ failure and the death of the food animal. Most typically, this is characterized by: peritonitis (bowel infection), cellulitis (skin infection), urinary tract infection (involving the kidneys and liver), osteomyelitis (bone infection), low platelet count and destruction of red blood cells in blood samples, elevated fibrin degradation products in the blood, and immature white blood cells in the blood. Second, the FDA has issued guidelines to the USDA regarding the need to limit anti-microbial agent use (Docket No. 1998-1146, CVM 200381, October 2003) in food animals in order to minimize the risk of increased pool of Antimicrobial Resistant (“AR”) pathogens in the food chain that could adversely affect humans.

Specific guidelines have been put in place by the FDA regarding the limitation of use of oxytetracycline, virginiamycin, bacitracin, flavomycin, tylosine and sulphaquinoxalines, due to the risk of increasing AR in humans consuming the residues of antimicrobial agents in food animals (beef and dairy cattle, poultry, swine). Because the agricultural industry is limited as to what steps it can use to therapeutically treat infected food animals, in order to minimize AR in consuming humans, there is a definite need for animal health products that effectively treat and/or prevent the incidence sepsis in food animals, while minimizing the risk of AR pathogens in the food chain. In addition, there is a need for compositions that treat and/or prevent the incidence of sepsis in humans.

### SUMMARY OF THE INVENTION

Electrolyzed saline solutions suitable for the treatment or prevention of sepsis are disclosed.

In one embodiment, a method for treating or preventing sepsis in an animal, comprising administering an electrolyzed saline solution to the animal is provided. In one embodiment, the electrolyzed saline solution comprises ozone and one or more active species selected from the group consisting of: active chlorine species, active oxygen species, and active hydrogen species, or combinations thereof. In one embodiment, the electrolyzed saline solution comprises ozone and at least one active chlorine species. In one embodiment, the electrolyzed saline solution comprises ozone, at least one active chlorine species, at least one active oxygen species, and at least one active hydrogen species. Exemplary active species include, e.g., HOCl⁻¹, OCL⁻¹, Cl⁻¹, Cl₂, O₂³, O₃, and H₂O₂.

The electrolyzed saline solution can comprise any amount of ozone and active species suitable for treating or preventing sepsis in an animal, such as, e.g., about 0.1 ppm to about 100 ppm ozone and about 5 ppm to about 300 ppm of at least one active chlorine species. The solution can also comprise about 0.1 ppm to about 300 ppm of at least one active oxygen species, and/or about 5 ppm to about 300 ppm of at least one active hydrogen species. In one embodiment, the solution comprises about 0.1 ppm to about 30 ppm ozone and about 10 ppm to about 100 ppm of at least one active chlorine species, and can also comprise about 0.1 ppm to about 100 ppm of at least one active oxygen species, and/or about 10 ppm to about 100 ppm of at least one active hydrogen species. In one embodiment, the solution comprises about 9 ppm to about 15 ppm ozone and about 10 ppm to about 80 ppm of at least one active chlorine species, and can also comprise about 0.1 ppm to about 80 ppm of at least one active oxygen species, and/or about 10 ppm to about 80 ppm of at least one active hydrogen species. In one embodiment, the electrolyzed saline solution comprises less than about 0.8 ppm ozone and about 10 ppm to about 80 ppm of at least one active chlorine species, such as, e.g., about 55 ppm to about 80 ppm of at least one active chlorine species. In yet another embodiment, the solution comprises about 0.30 to about 0.7 ppm ozone and about. 10 ppm to about 80 ppm of at least one active chlorine species.

In one embodiment, the administration of the electrolyzed saline solution to a food animal minimizes the risk of increased antimicrobial resistant pathogens in the food chain. In one embodiment, the administration of the electrolyzed saline solution to a human minimizes the risk of increased antimicrobial resistant pathogens in the food chain.

### BRIEF DESCRIPTION OF THE DRAWINGS

Many aspects of the invention can be better understood with reference to the following drawings:

FIG. 1A is a photograph showing the lung histopathology of a mouse treated only with saline.

FIG. 1B is a photograph showing the lung histopathology of a mouse treated with bacteria only.

FIG. 1C is a photograph showing the lung histopathology of a mouse treated with bacteria in the peritoneum and then with 100% MDI-P in the peritoneal cavity.

FIG. 2A is a photograph showing the spleen histopathology of a mouse treated with bacteria and with 100% MDI-P.

FIG. 2B is a photograph of an MDI-P treated spleen showing that the red pulp cord meshwork contains a large number of erythrocytes and platelets (arrow).

FIG. 2C is a photograph showing the spleen histopathology of a mouse treated with bacteria only.

FIG. 2D is a photograph showing the spleen histopathology of a mouse treated with bacteria and with 25% MDI-P.

FIG. 3A is a photograph showing the liver histopathology of a mouse that received saline only.

FIG. 3B is a photograph showing the liver histopathology of a mouse treated with bacteria and 100% MDI-P.

FIG. 3C is a photograph showing the liver histopathology of a mouse treated with bacteria and 50% MDI-P.

FIG. 3D is a photograph showing the liver histopathology of a mouse treated only with bacteria.

FIG. 4A is a photograph showing the kidney histopathology of a mouse treated with 100% MDI-P after inoculation with bacteria.

FIG. 4B is a photograph showing the kidney histopathology of a mouse treated with bacteria only.

FIG. 5A is a photograph showing the heart histopathology of a mouse treated with 100% MDI-P after bacterial infection.

FIG. 5B is a photograph showing the changed vasculature in a mouse treated with bacteria and 25% MDI-P.

### DETAILED DESCRIPTION OF THE INVENTION

It has now been found that saline solutions, which have been subjected to electrolysis to produce ozone and active products, such as, e.g., active chlorine products, active oxygen products, and active hydrogen products, are useful for the treatment and prevention of sepsis in an animal, such as a mammal, such as a human. Furthermore, such electrolyzed saline solutions are useful for treating and/or preventing the incidence of sepsis in food animals while minimizing the risk of AR pathogens in the food chain.

Composition

The present invention provides electrolyzed saline solutions. In one embodiment, the electrolyzed saline solution comprises ozone and one or more active species selected from the group consisting of: active chlorine species, active oxygen species, and active hydrogen species, or combinations thereof. In one embodiment, the electrolyzed saline solution comprises ozone and at least one active chlorine species. In one embodiment, the electrolyzed saline solution comprises ozone, at least one active chlorine species, at least one active oxygen species, and at least one active hydrogen species. Exemplary active species include, e.g., HOCl⁻¹, OCL⁻¹, Cl⁻¹, Cl₂, O₂³, O₃, and H₂O₂.

The electrolyzed saline solution can comprise any amount of ozone and active species suitable for treating or preventing sepsis in an animal, such as, e.g., about 0.1 ppm to about 100 ppm ozone and about 5 ppm to about 300 ppm of at least one active chlorine species. The solution can also comprise about 0.1 ppm to about 300 ppm of at least one active oxygen species, and/or about 5 ppm to about 300 ppm of at least one active hydrogen species. In one embodiment, the solution comprises about 0.1 ppm to about 30 ppm ozone and about 10 ppm to about 100 ppm of at least one active chlorine species, and can also comprise about 0.1 ppm to about 100 ppm of at least one active oxygen species, and/or about 10 ppm to about 100 ppm of at least one active hydrogen species. In one embodiment, the solution comprises about 9 ppm to about 15 ppm ozone and about 10 ppm to about 80 ppm of at least one active chlorine species, and can also comprise about 0.1 ppm to about 80 ppm of at least one active oxygen species, and/or about 10 ppm to about 80 ppm of at least one active hydrogen species. In one embodiment, the electrolyzed saline solution comprises less than about 0.8 ppm ozone and about 10 ppm to about 80 ppm of at least one active chlorine species, such as, e.g., about 55 ppm to about 80 ppm of at least one active chlorine species. In yet another embodiment, the solution comprises about 0.30 to about 0.7 ppm ozone and about 10 ppm to about 80 ppm of at least one active chlorine species.

In one embodiment, the electrolyzed saline solution comprises about 5 ppm to about 80 ppm of at least one active hydrogen species, such as, e.g., about 10 ppm of at least one active hydrogen species. In one embodiment, the active hydrogen species is less than about 15 ppm. In one embodiment, the active hydrogen species is hydrogen peroxide.

In one embodiment, the electrolyzed saline solution comprises about 5 ppm to about 300 ppm of at least one active chlorine species, such as, e.g., free chlorine. In one embodiment, the electrolyzed saline solution comprises about 10 ppm to about 80 ppm of at least one active chlorine species. In one embodiment, the electrolyzed saline solution comprises about 55 ppm to about 80 ppm of at least one active chlorine species. In one embodiment, the electrolyzed saline solution comprises about 60 ppm of at least one active chlorine species. In one embodiment, the electrolyzed saline may or may not comprises ozone.

In one embodiment, the electrolyzed saline solution comprises about 5 ppm to about 300 ppm total chlorine. In one embodiment, the electrolyzed saline solution comprises about 10 ppm to about 80 ppm total chlorine, such as, e.g., about 50 ppm to about 70 ppm total chlorine, such as, e.g., about 60 ppm total chlorine.

In one embodiment, the electrolyzed saline solution has a redox potential of about 500 mV to about 1500 mV. In one embodiment, the electrolyzed saline solution has a redox potential of about 800 mV to about 900 mV, such as, e.g., about 850 mV.

In one embodiment, the concentration of active species can be expressed in terms of reactive oxygen species (ROS). In one embodiment, the electrolyzed saline solution comprises a sum of ROS activity from about 5 to about 100,000 μM relative to AAPH (2,2′-azobis(2-aminopropane) dihydrochloride) and/or allowing for inhibition of indicator strains within this range. In one embodiment, the electrolyzed saline solution has a ROS of from about 0.3 mM to about 10 mM ROS, such as, e.g., from about 0.5 mM to about 5 mM ROS, such as, e.g., from about 1 mM ROS to about 3 mM ROS, such as, e.g., from about 1 to about 2.5 mM ROS.

In one embodiment, the electrolyzed saline solution has an osmolarity from about 100 mOsm to about 500 mOsm, such as, e.g, about 200 mOsm to about 300 mOsm, such as, e.g., about 284 mOsm.

In one embodiment, the electrolyzed saline solution comprises less than about 4,000 ppm sodium, such as, e.g., about 3,900 ppm sodium.

The pH of the electrolyzed saline solution can be any pH suitable for the method of use. In one embodiment, the pH is from about 6.5 to about 8, such as, e.g., about 6.75 to about 7.5, about 7 to about 7.6, or about 7.2 to about 7.8. For example, in one embodiment the pH of the solution is in the range from about 7.2 to about 7.6. In one embodiment, the pH of the solution is in the range from about 6.75 to about 7.5. In one embodiment, when the solution is used for intravenous administration, the pH of the solution is in the range from about 7.35 to about 7.45 which is the pH range of human blood.

In one embodiment, the electrolyzed saline solution comprises less than about 0.8 ppm ozone and from about 55 ppm to about 80 ppm of at least one active chlorine species, such as, e.g., free chlorine. In one embodiment, the electrolyzed saline solution further comprises less than about 15 ppm active hydrogen species, such as, e.g., hydrogen peroxide. In one embodiment, the electrolyzed saline solution has a ROS from about 0.3 mM to about 10 mM, a pH from about 6.75 to about 7.5, and/or an osmolarity from about 200 mOsm to about 300 mOsm, such as, e.g., about 284 mOsm.

For purposes of this invention, the term “active species” or “active product” means any species or product resulting from the subjection of a saline solution to electrolysis, such as, e.g., an active chlorine species, an active oxygen species, and an active hydrogen species. The term “active species” or “active product” can mean one active species or a combination of active species. The term “active chlorine agent or species,” means one or more of any active form of chlorine resulting from the subjecting of a saline solution to electrolysis which can be measured by a chlorine selective electrode, such as, e.g., free chlorine, hypochlorous acid and the hypochlorite ion. The term “active oxygen agent or species” means one or more of any active form of oxygen resulting from the subjecting of a saline solution to electrolysis, such as, e.g., O₂³. The term “active hydrogen agent or species” means one or more of any active form of hydrogen resulting from the subjecting of a saline solution to electrolysis, such as, e.g., H₂O₂.

The composition can also comprise other products of the electrolysis reaction including ions selected from the group consisting of hydrogen, sodium and hydroxide ions. The interaction of the electrolysis products can result in a solution comprising bioactive atoms, radicals or ions selected from the group consisting of chlorine, ozone, hydroxide, hypochlorous acid, hypochlorite, peroxide, oxygen and perhaps others along with corresponding amounts of molecular hydrogen and sodium and hydrogen ions. In one embodiment, the electrolyzed saline solution comprises HOCl⁻¹, OCL⁻¹, Cl⁻¹, Cl₂, O₂³, O₃, and H₂O₂. The HOCl⁻, OCL⁻¹, Cl⁻¹, Cl₂, O₂³, O₃, and H₂O_{2.} can be present in any suitable amount, such as, e.g., about 0.1 ppm to about 300 ppm for each active species.

The electrolyzed saline solution can be prepared from any sterile saline solution suitable for producing the desired electrolyzed saline solution upon electrolysis. In one embodiment, the saline solution has an initial concentration from about 0.05% to about 10.0% NaCl. In one embodiment, the saline solution has an initial concentration from about 0.1% to about 5.0% NaCl. In another embodiment, the saline solution has an initial concentration from about 0.15% to about 1% NaCl, such as, e.g., an initial concentration from about 0.25% to about 1.0% NaCl. In one embodiment, the saline solution has an initial concentration of about 0.9% NaCl. In one embodiment, the saline solution has an initial concentration of about 0.45% NaCl. In one embodiment, the saline solution has an initial concentration of about 0.215% NaCl.

The saline solution can be subjected to electrolysis at any suitable voltage, current, and time to produce an appropriately electrolyzed solution. Suitable methods and equipment for performing the electrolysis are described in, e.g., U.S. Pat. Nos. 5,334,383; 5,507,932; 5,560,816; 5,622,848; 5,674,537; 5,731,008; 6,007,686; and 6,117,285, herein incorporated by reference. In one embodiment, the electrolysis reaction is performed at ambient temperatures.

In one embodiment, the saline solution is diluted with sterile distilled water to the desired concentration, such as, e.g., concentrations from about 0.05% to about 10.0% NaCl (e.g., about 0.1% to about 5.0% NaCl; about 0.15% to about 1% NaCl; or about 0.25% to about 1.0% NaCl). The diluted saline solution is then subjected to electrolysis at sufficient voltage, amperage and time to produce an electrolyzed solution comprising the desired concentrations of ozone and active chlorine, active oxygen, and/or active hydrogen species. The electrolysis reaction can be carried out at any suitable temperature. In one embodiment, the electrolysis reaction is carried out at ambient temperatures.

Obviously, the voltage and amperage to be used and the time of electrolysis is subject to many variables, i.e. the size and composition of the electrodes, the volume and/or concentration of saline being electrolyzed. For large electrodes or saline volumes or higher concentrations of saline solutions the voltage, amperage or time may be higher and/or longer. It is the generation of the desired concentration of ozone and active chlorine, active oxygen, and/or active hydrogen species that is important. According to Faraday's laws of electrolysis, the amount of chemical change produced by a current is proportional to the quantity of electricity passed. Also, the amounts of different substances liberated by a given quantity of electricity are proportional to the chemical equivalent weights of those substances.

Therefore, to generate an electrolyzed saline having the desired concentrations of ozone and active chlorine, active oxygen, and/or active hydrogen species from saline solutions having a saline concentration of less than about 1.0%, voltage, amperage and time parameters appropriate to the electrodes and solution are required to produce an electrolyzed solution comprising from about 0.1 to 100 ppm of ozone, such as, e.g., about less than 0.8 ppm ozone, and a free chlorine content from about 5 to 300 ppm, such as, e.g., about 55 ppm to about 80 ppm free chlorine. In one embodiment, the treatment produces an electrolyzed solution comprising from about 0.1 to about 50 ppm of ozone and a free chlorine content from about 10 to about 100 ppm. In a further embodiment, the treatment produces an electrolyzed solution comprising from about 0.1 to about 30 ppm of ozone and a free chlorine content from about 20 to about 60 ppm. In another embodiment, the treatment produces an electrolyzed solution comprising from about 0.1 to about 1.0 ppm ozone and a free chlorine content from about 50 to about 70 ppm. In yet another embodiment, the treatment produces an electrolyzed saline solution comprising less than about 0.8 ppm ozone and from about 55 to about 80 ppm of free chlorine.

The concentration of the active species can be measured by any suitable manner, such as, e.g., titration; methods described in Hoigne and Bader, Water Research, 5:449-456 (1981); HACH colorimeter Indigo method, or any other suitable method. Similarly, pH and redox potential can also be measured by any suitable method.

For in vitro use these solutions can be utilized without further modification or they can be adjusted as desired with saline or other solutions. For in vivo use these solutions can be utilized without further modification or they can be adjusted as desired with saline or other solutions. Prior to in vivo use, this solution may be adjusted or balanced to an isotonic saline concentration with sufficient hypertonic saline, e.g. 5% hypertonic saline solution.

In one embodiment, the electrolyzed saline solution is filtered prior to measurement. In one embodiment, the electrolyzed saline solution is filtered prior to administration or use.

In one embodiment, an electrolyzed saline solution can be obtained by subjecting about a 0.33% (about one third physiologically normal) saline solution to electrolysis for about 5 to 15 minutes. The voltage between the electrodes was maintained in the range of about 10 to 20 volts at a current in the range of about 5 to 20 amps, such that the freshly prepared electrolyzed saline when balanced or normalized with sterile 5% saline contained about 10 ppm to about 200 ppm of active chlorine species, such as, e.g., about 55 ppm to about 80 ppm of active chlorine species, along with about 0.1 to 30 ppm of ozone, such as, e.g., about less than 0.8 ppm ozone, and corresponding amounts of molecular hydrogen and sodium and hydrogen ions.

In one embodiment, the electrolyzed saline solution remains stable in sealed sterile containers for a suitable period of time, such as, e.g., about 6 months, about one year or about 18 months.

Dosage and Dosage Forms

Particular dosages and methods of administration, as well as additional components to be administered, can be determined by those skilled in the art using the information set forth herein and set forth in the U.S. patent documents previously incorporated herein by reference.

An effective amount of the electrolyzed saline solution can be administered by any appropriate mode, e.g., intranasally, parenterally, e.g., intravenously (i.v.) or intraperitoneally (i.p.), orally, vaginally or rectally and may vary greatly according to the mode of administration, condition being treated, the size of the warm-blooded animal, etc. In one embodiment, the electrolyzed saline solution is administered in the form of an inhaler.

The electrolyzed saline solution of the present invention can be prepared in any suitable dosage form. In one embodiment, the electrolyzed saline solution can be formulated as a single pharmaceutical composition or as independent multiple pharmaceutical dosage forms. Pharmaceutical compositions according to the present invention include those suitable for intranasal, topical, oral, rectal, buccal (for example, sublingual), or parenteral (for example, intravenous) administration, although the most suitable route in any given case will depend on the nature and severity of the condition being treated as well as by the type of mammal being treated. Of course, intravenous or intraperitoneal injections are typically suitable for delivering more active solution into the bloodstream of the animal. Such delivery can be suitable to quickly deliver the electrolyzed saline solution if the animal is suffering from a potentially fatal infection or disease state.

The electrolyzed saline solution of the present invention can be administered to any animal, such as, e.g., a mammal. In one embodiment, the mammal is a human. In one embodiment, the electrolyzed saline solution is used in a veterinary application for administration to mammals, reptiles, birds, exotic animals and farm animals, including, e.g., a monkey, or a lemur, a horse, a cow, a chicken, a pig, a dog, a cat, or a rodent, e.g., a rat, a mouse, a squirrel or a guinea pig. In one embodiment, the mammal is a food animal, such as any animal suitable for serving as food to a human or another animal, e.g., a cow, a calf, a steer, a chicken, a turkey, a goose, a duck, a sheep, or a pig.

For an animal, an intravenous injection dosage of the electrolyzed saline solution may vary from between about 0.01 ml/kg/day body weight to about 10 ml/kg/day body weight. In one embodiment, the i.v. injection dosage of the electrolyzed saline solution is between about 0.25 to about 4 ml/kg/day body weight, such as, e.g., from about 0.5 to 3.0 ml/kg/day, such as, e.g., from about 0.25, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 ml/kg/day body weight. The doses can be divided into smaller doses and administered two or more times per day or may be administered in a single dose. The regimen can vary according to the indication being treated. For example, it may be advantageous to administer the electrolyzed saline solution for several days followed by a rest period and repeating the cycle for as long as necessary or as indicated by test results. A typical regimen might be five days of treatment followed by two days rest with the cycle repeated for two months. Depending on clinical status or laboratory tests, this regimen may be reduced to, e.g. three days of treatment per week for six weeks. These regimens are exemplary only and are not meant to be limiting as any number or variation might be dictated according to circumstances.

In certain situations where it may be desired to utilize higher concentrations of chlorine and oxygen active agents produced from the electrolysis of a saline solution, it may be desirable to concurrently administer in vivo or subject a solution in vitro to modulating or moderating chemicals. These modulating chemicals are administered before, concurrent with or after the electrolyzed saline and may be administered in any suitable manner, such as, e.g., intravenously, parenterally, intranasally, or orally. As used herein the terms “moderating”, “modulating” and “neutralizing” agents may be used interchangeably.

The modulating chemicals are enzymes or reducing agents that interact with and reduce the active microbicidal agents to innocuous compounds. The enzymes are inclusive of, but not limited to, the superoxide dismutases (SOD), catalase and glutathione peroxidase. These oxygen radicals are converted to hydrogen peroxide by Cu/Zn activated superoxide dismutases (SOD) in the cells. In a properly functioning system the hydrogen peroxide is then converted to oxygen and water by a catalase. If the hydrogen peroxide and the superoxide radical are allowed to combine, the more deadly hydroxide radical is formed.

Administration

The electrolyzed saline solution of the present invention can be suitable for the treatment of bacterial, viral and fungal related syndromes and immunological disorders. Examples of such syndromes and/or immunological disorders for which either in vitro or in vivo treatment could be beneficial are Epstein-Barr virus, hepatitis A, B and C, rhinovirus, rubeola, rubella, parvovirus, papilloma virus, influenza and parainfluenza viruses, enteroviruses; Herpes simplex viruses; Varicella-zoster viruses, Adenoviruses, respiratory syncytial viruses, alphaviruses, flaviviruses, retroviruses (including AIDS and AIDS related syndromes), bacteremia, septicemia, fungal infections, parasitic infections (nematodes, trematodes, protozoal, e.g., Cryptosporidium helminthic), mycobacterial infections, bacterial Gram positive and Gram negative superficial and systemic infections and other viral, bacterial and/or fungal associated diseases.

There are also situations where fluids can be beneficially treated in vitro, to purify, decontaminate, or otherwise render such fluid acceptable for administration to a warm-blooded host. For example, the blood supply taken from donors at blood banks has been found on occasion to be contaminated by the HIV virus and other organisms such as hepatitis A, B and C viruses, CMV (cytomegalovirus), and bacteria (such as Yersinia). Any treatment of whole blood, plasma or cell isolates to render them benign from infectious organisms without destroying the therapeutic characteristics of such fluids would be very beneficial.

The electrolyzed saline solution is suitable for the treatment or prevention of sepsis, as described herein. In the context of the present invention, the word “treatment” can mean any positive change in the symptoms or pathology of the treated disorder, such as, e.g., the complete eradication of the disorder, a reduction in the severity of symptoms, extension of the patient's life, and/or a discontinuance in the negative progression of the disorder.

The electrolyzed saline solution can comprise any amount of ozone and active species suitable for treating or preventing sepsis in an animal, such as, e.g., about 0.1 ppm to about 100 ppm ozone and about 5 ppm to about 300 ppm of at least one active chlorine species. The solution can also comprise about 0.1 ppm to about 300 ppm of at least one active oxygen species, and/or about 5 ppm to about 300 ppm of at least one active hydrogen species. In one embodiment, the solution comprises about 0.1 ppm to about 30 ppm ozone and about 10 ppm to about 100 ppm of at least one active chlorine species, and can also comprise about 0.1 ppm to about 100 ppm of at least one active oxygen species, and/or about 10 ppm to about 100 ppm of at least one active hydrogen species. In one embodiment, the solution comprises about 9 ppm to about 15 ppm ozone and about 10 ppm to about 80 ppm of at least one active chlorine species, and can also comprise about 0.1 ppm to about 80 ppm of at least one active oxygen species, and/or about 10 ppm to about 80 ppm of at least one active hydrogen species. In one embodiment, the electrolyzed saline solution comprises less than about 0.8 ppm ozone and about 10 ppm to about 80 ppm of at least one active chlorine species, such as, e.g., about 55 ppm to about 80 ppm of at least one active chlorine species. In yet another embodiment, the solution comprises about 0.30 to about 0.7 ppm ozone and about 10 ppm to about 80 ppm of at least one active chlorine species.

In one embodiment, the active chlorine species comprises at least one of an active chlorine species selected from the group consisting of: free chlorine, hypochlorous acid and hypochlorite ion. In one embodiment, the active oxygen species is O₂³. In one embodiment, the active hydrogen species is H₂O₂. In one embodiment, the solution is prepared by subjecting a 1% or less saline solution, such as, e.g., 0.9% NaCl (w/vol), 0.45% NaCl (w/vol), and 0.215% NaCl (wt/vol), to electrolysis under conditions sufficient to produce the desired active ingredients.

The electrolyzed saline solution can be administered in any suitable manner. In one embodiment, the method for treating or preventing sepsis in an animal comprises administering the electrolyzed saline solution to by intravenous or intraperitoneal injection to an animal, such as, e.g., a human or a food animal. The food animal can be any animal suitable for serving as food to a human or another animal, such as, e.g., a cow, a calf, a steer, a chicken, a turkey, a goose, a duck, a sheep, or a pig.

In one embodiment, the administration of the electrolyzed saline solution to a food animal minimizes the risk of antimicrobial-resistant pathogens developing in the food chain. In one embodiment, the administration of the electrolyzed saline solution to a human minimizes the risk of the development of antimicrobial-resistant pathogens. Administration of the electrolyzed saline solution exhibits no apparent toxicity or tissue residue.

The invention is further illustrated by the following examples, which of course should not be construed as in any way limiting its scope.

### EXAMPLES

### Example 1

This example demonstrates the effect of electrolyzed saline administration on intraperitoneal infection. Specifically, the example demonstrates that administration of electrolyzed saline solution results in a sparing effect from a fatal dose of bacterium in induced sepsis.

Induced Sepsis Protocol

Mice (C₃H) were induced with B. Pseudomonas aeruginosa to have peritoneal sepsis, with resultant infection injury to the lung. Fox-Dewhurst, R., et al., Am. J Respir. Crit. Care Med., 155:2030-2040 (1997). Groups of animals (n=4 per group) were treated with doses of bacteria (108-1010 cfu/ml) in the peritoneum. Injection bacteria was prepared in saline at 10×10⁷ cfu/ml. 0.3 ml of bacterial solution was injected into the peritoneal cavity. After three minutes, 0.3 ml of saline or 0.5 ml of electrolyzed saline solution was injected intraperitoneally. A group of animals was treated with Gentamycin as a positive control to compare the effectiveness of 25%, 50%, and 100% electrolyzed saline solutions. This group of animals was treated at the time of bacterial inoculation with Gentamycin at a concentration of 1.0 mg/ml intraperitoneally.

Drug Treatment

Six study groups of mice were studied: saline control, Gentamycin positive control, bacteria plus 25% electrolyzed saline solution, bacteria plus 50% electrolyzed saline solution, bacteria plus 100% electrolyzed saline solution, and bacteria plus saline. 24 mice were divided into 6 groups as indicated in the following table.

TABLE 1

| | Group | | | | | |
|---|---|---|---|---|---|---|
| | 1 | 2 | 3 | 4 | 5 | 6 |
| Treatment | Control | Bacteria + | Bacteria + | Bacteria + | Bacteria + | Bacteria + |
| | Saline | MDI 25% | MDI 50% | MDI 100% | Saline | Gentamicin |
| N | 4 | 4 | 4 | 4 | 4 | 4 |

Histopathology

All of the animals were taken for necropsy. Tissue organs were selected and excised for histology. All tissues were fixed in 10% neutral buffered formalin. After 24 hours fixation, tissues were dehydrated and embedded into paraffin blocks. The paraffin blocks were sectioned and 2 levels of each block separated by 500 mm distance were selected and 2 slides were made of each level. Slides were stained with hematoxylin and eosin to examine the histopathology.

TABLE 2
Survival Table

| | Treatment | | | | | |
|---|---|---|---|---|---|---|
| | Saline | 25% | 50% | 100% | Bact. | Gentamycin |
| n | 4 | 4 | 4 | 4 | 4 | 4 |
| 24 hrs | 4 | 4 | 4 | 4 | 0 | 4 |
| 48 hrs | 4 | 0 | 0 | 4 | 0 | 4 |
| 72 hrs | 4 | 0 | 0 | 2 | 0 | 2 |

All animals were dead at night and autopsy was done the next morning. Some of the animals treated with 25% and 50% electrolyzed saline solution already smelled of deterioration after 48 hours.

Severe peritoneal infection with P. aeruginosa may be associated with bacteremia; however, it rarely causes blood stream infection. In this study, the animals showed signs of systemic sepsis as would be seen with other gram-negative pathogens. P. aeruginosa has the ability to invade epithelial cells to cause massive cell injury and necrosis in various organs as seen in the lungs, livers, kidneys, spleens, and hearts with the bacterial inoculation groups. Within 24 hours, all control animals treated with bacteria alone were dead and the tissues were taken out for examination. The groups treated with electrolyzed saline solutions at this time period were still active, only one individual observed was less active. At 48 hours, the 25% electrolyzed saline solution and 50% electrolyzed saline solution-treated groups were all dead.

The bodies of some of the mice in the 25% electrolyzed saline solution group were deteriorated and beginning to decompose at 48 hours. In the 50% electrolyzed saline solution group, the bodies still appeared normal with no signs of decomposition. At 48 hours, the 100% electrolyzed saline solution and Gentamicin group were still normal with no sign of sickness. At 72 hours, the saline group of 4 animals was normal, but 2 animals had died in the 100% electrolyzed saline solution group and in the Gentamicin group. In these two groups, a 50% survival rate resulted by treatment.

Lung

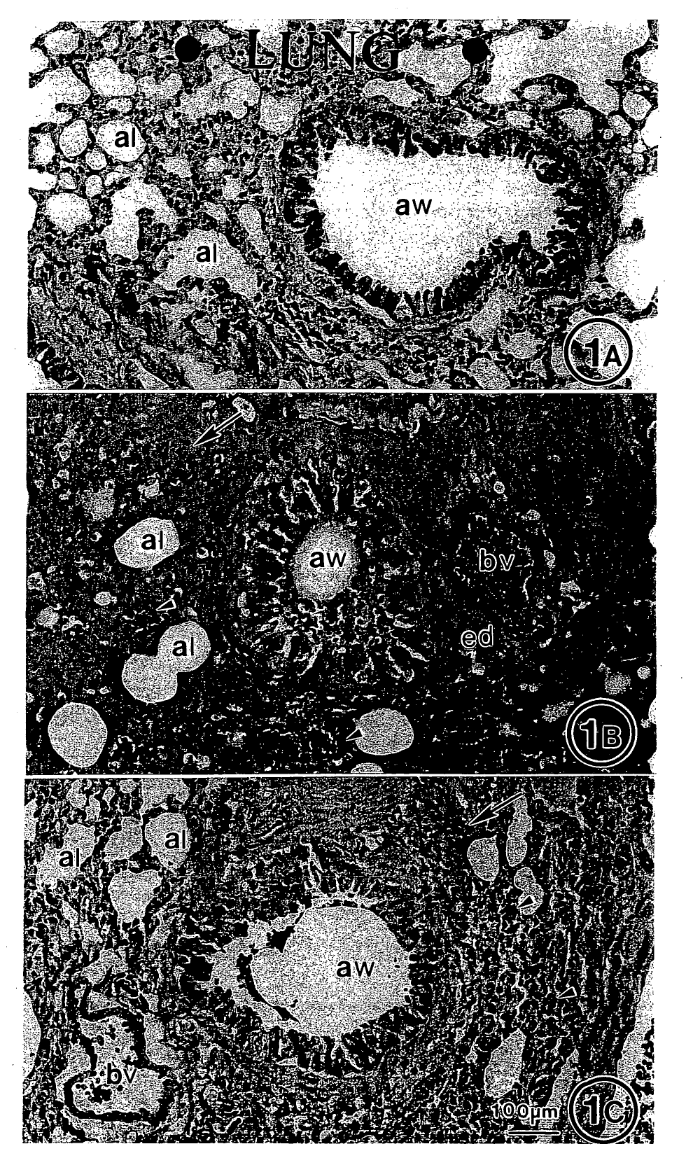

Saline control mice received no bacteria, only 0.3% ml of saline injected into the peritoneum. After 3 days (72 hours) the lung had a normal appearance, the alveoli (AL) were clean without edema or infiltrations, the airway was normal. (FIG. 1A).

FIG. 1B is a photograph showing the lung histopathology of a mouse treated with bacteria only. The lung shows overwhelming evidence of edema (Ed). The alveoli (AL) are consulted with hemorrhage red blood cells (arrowheads). The airway is constricted with amorphous substances and the blood vessel (BV) is consolidated with red blood cells. Inflammatory cells have infiltrated into the lung tissues (arrow).

FIG. 1C is a photograph showing the lung histopathology of a mouse treated with bacteria in the peritoneum and then with 100% MDI-P in the peritoneal cavity. The lung shows fairly normal alveolar appearance (Al) with only a little bit of red blood cell fill in the small blood vessels (arrowheads). There are fewer inflammatory cell infiltrates in the lung tissues (arrow) that in untreated mouse lung.

Spleen

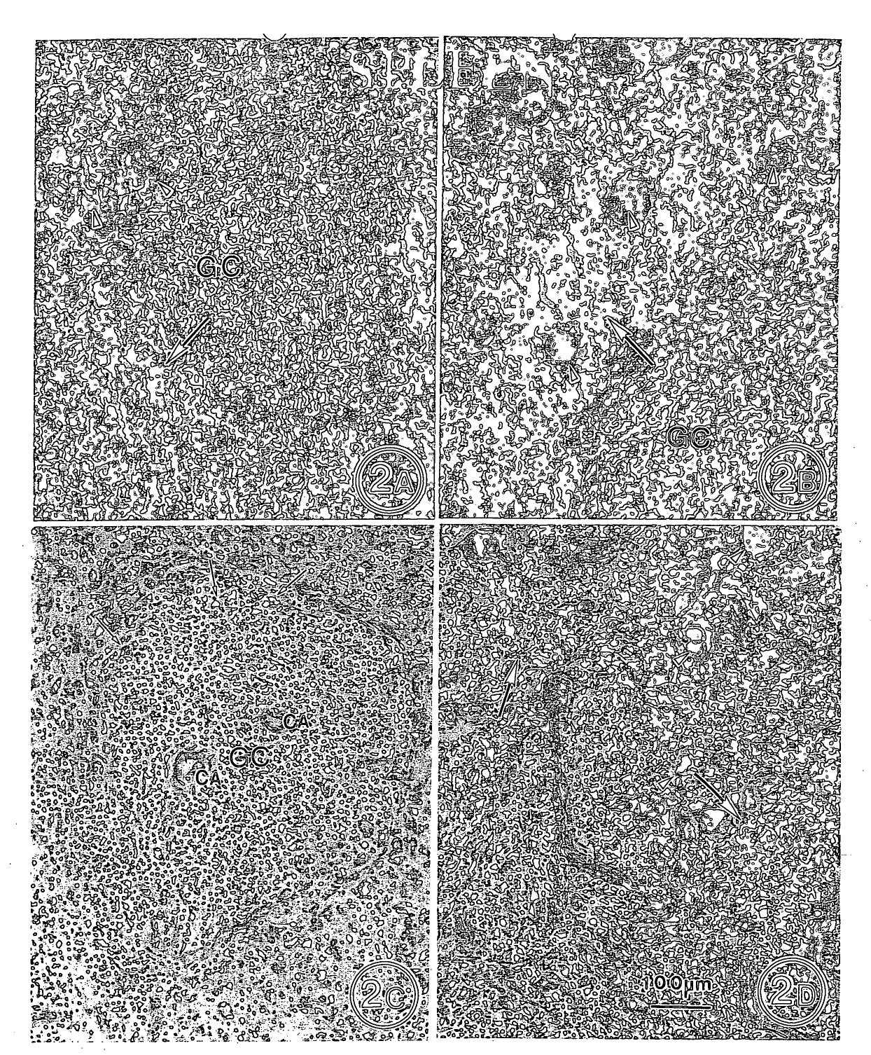

FIG. 2A is a photograph showing the spleen histopathology of a mouse treated with bacteria and with 100% MDI-P. The photograph shows that the spleen stores a large amount of blood cells similar to a normal spleen. The germinal center (GC) appears spherical, very much like the splenic nodule. The proliferation of B lymphoblasts is largely restricted to the GC. The subsegment differentiation into plasma cells occurs at the periphery of the GC next to the red pulp (arrowhead). The red pulp region presents a large amount of blood (arrow).

FIG. 2B is a photograph of an MDI-P treated spleen showing that the red pulp cord meshwork contains a large number of erythrocyte and platelets (arrow). There are islands of hemopoietic tissue in the red pulp that contains erythrocytes, myeloblasts and megakaryocytes (arrowheads).

FIG. 2C is a photograph showing the spleen histopathology of a mouse treated with bacteria only. The photograph shows that the germinal center (GC) iof the splenic nodule is full of B lymphocytes and B lymphoblasts. Many plasma cells are located in the periphery of the GC (arrow). The spherical GC contains the central artery (Currently Amended) in this section. There is no blood in the red pulp as seen in FIGS. 2A and 2B, which indicates that the hemopoietic process in the spleen was blocked by the bacterial invasion.

FIG. 2D is a photograph showing the spleen histopathology of a mouse treated with bacteria and with 25% MDI-P. The photograph shows that the red pulp has less blood as compared to the 100% MDI-P treated mouse shown in FIGS. 2A and 2B (arrows). There are many large cells in the peripheric process (arrowheads).

Treatment with 100% electrolyzed saline solution retains these morphologic characteristics in the spleen histology.

Liver

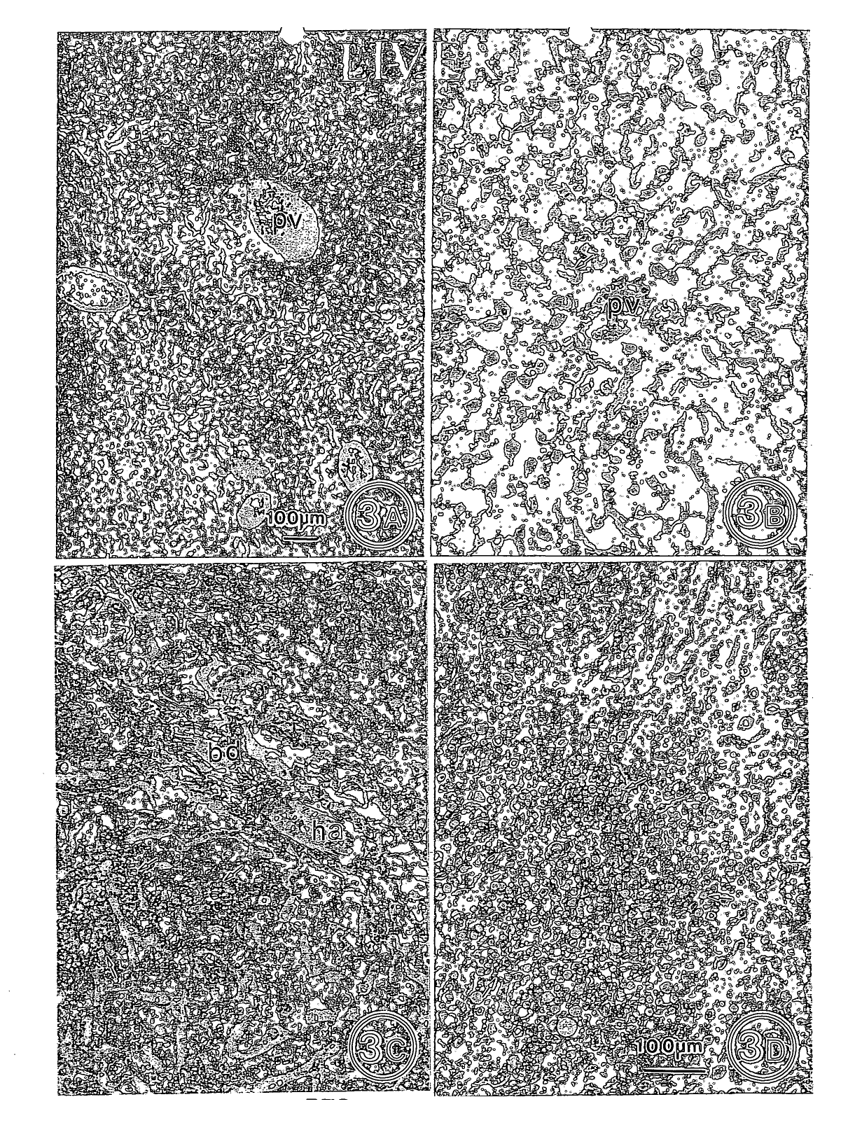

The liver is an accessory gland of the gastro-intestinal tract but it has a remarkable diversity of other function unrelated to alimentation. The liver is the site of synthesis of plasma proteins. Other proteins produced in the liver include fibrinogen, thrombin, and faction III, substances essential for blood clotting. Analysis of the liver of a mouse receiving a 0.5 ml saline i.p. showed normal liver cells. (FIG. 3A). Liver lobules also appeared typical.

FIG. 3A is a photograph showing the liver histopathology of a mouse that received saline only. A peripheral portion of a liver lobule is showing a typical portal vein (PV) and small bile ducts.

FIG. 3B is a photograph showing the liver histopathology of a mouse treated with bacteria and 100% MDI-P. The photograph shows that the hepatic lobules show a portal view (PV) and that the sinusoids contained few neutrophils (arrows). The hepatic cells appear normal.

FIG. 3C is a photograph showing the liver histopathology of a mouse treated with bacteria and 50% MDI-P. The photograph shows that the liver tissue has signs of focal lesions (arrows). The lesion is a vacuolization of liver cells, as seen in the fatty liver. There is damage in the hepatic artery (ha) endothelia and the surrounding intestine. The bile duct (bd) seems normal.

FIG. 3D is a photograph showing the liver histopathology of a mouse treated only with bacteria. The photograph shows injury by fatty accumulation in the hepatic tissue vacuolization (arrows).

Kidney

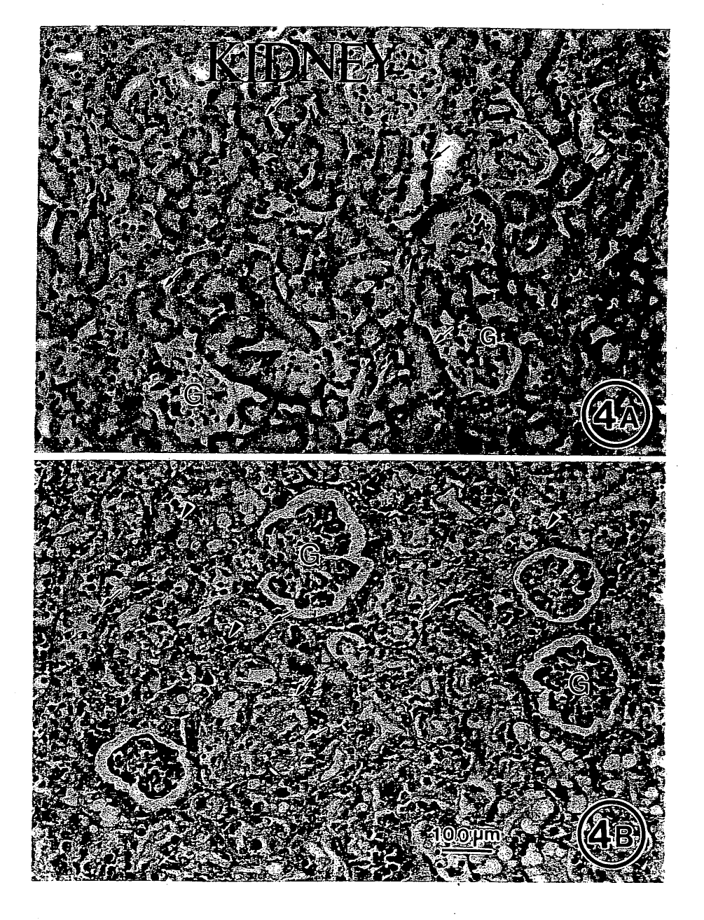

Analysis of the kidney of a mouse treated with 100% electrolyzed saline solution after inoculation with bacteria showed the critical labyrinth consists mainly of the proximal convoluted tubules (FIG. 4A). The figure demonstrates that the critical labyrinth consists mainly of the proximal convoluted tubules (arrows). The cells appear as eosinophilic cuboidal with round nuclei. The glomeruli are normal (G) and the Bowman's capsule displayed no injury.

FIG. 4B is a photograph showing the kidney histopathology of a mouse treated with bacteria only. The renal tubules are necrotic (arrows) and many red blood cells are observed in the interstitial region (arrowhead). The glomeruli (G) are reduced and filled with RBC in the capillaries.

Heart

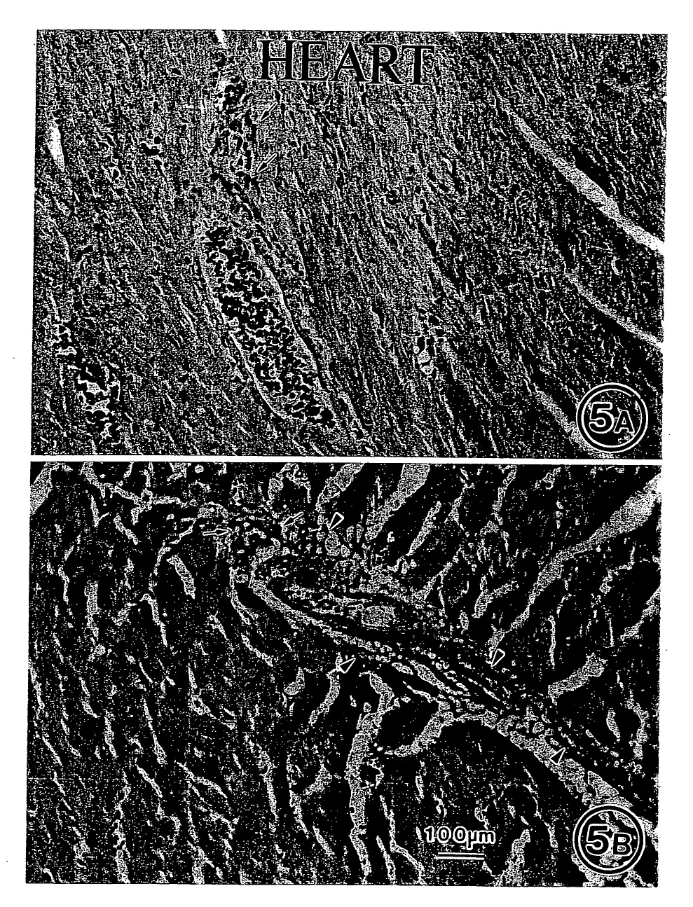

Mice treated with 100% electrolyzed saline solution after bacterial infections had normal myocytes in the heart muscle tissues. Only a few cell infiltrations occurred in the vascular region (FIG. 5A). FIG. 5A is a photograph showing the heart histopathology of a mouse treated with 100% MDI-P after bacterial infection. The photograph shows normal myocytes in the heart muscle tissues. Only a few cell infiltrations occurred in the vascular region (arrows).

In mice treated with 25% electrolyzed saline solution after bacterial inoculation, the vasculature are obviously changed. (FIG. 5B). FIG. 5B is a photograph showing the changed vasculature in a mouse treated with bacteria and 25% MDI-P (arrowheads).

As demonstrated by this example, electrolyzed saline solution-treated mice exhibited improved lung histology, spleen histopathology, liver histopathology, kidney histopathology and heart histopathology compared to untreated saline plus bacteria control mice. In all electrolyzed saline solution treatment groups, PMN migration was reduced, with resultant reduced local infection. Further, a 50% sparing effect (i.e., 50% survival) at the 100% electrolyzed saline solution dose level was found, comparable to Gentamicin. No apparent toxicity due to electrolyzed saline solution was exhibited in any of the study mice groups.

All publications, patents, and patent application documents are incorporated by reference herein in their entirety, as though individually incorporated by reference. The invention has been described with reference to various specific and preferred embodiments and techniques. However, it should be understood that many variations and modifications can be made while remaining within the spirit and scope of the invention.

I claim:

1. A method for treating or preventing sepsis in an animal, comprising administering an electrolyzed saline solution to the animal.

2. The method of claim 1, wherein the electrolyzed saline solution comprises ozone and one or more active species selected from the group consisting of: active chlorine species, active oxygen species, and active hydrogen species.

3. The method of claim 2, wherein the active chlorine species comprises at least one of an active chlorine species selected from the group consisting of: free chlorine, hypochlorous acid and hypochlorite ion.

4. The method of claim 1, wherein the solution is prepared by subjecting a 1% or less saline solution to electrolysis under conditions sufficient to produce the desired active ingredients.

5. The method of claim 4, wherein the solution is prepared using a saline solution with a starting sodium chloride solution selected from the group consisting of: 0.9% NaCl (w/vol), 0.45% NaCl (w/vol), and 0.215% NaCl (wt/vol).

6. The method of claim 1, wherein the solution comprises HOCl⁻¹, OCL⁻¹, Cl⁻¹, Cl₂, O₂³, O₃, and H₂O₂.

7. The method of claim 1, wherein the solution is administered by intravenous or intraperitoneal injection.

8. The method of claim 1, wherein the solution is administered by injection to a food animal at a dosage from about 0.25 mi/kg/day body weight to about 4 mi/kg/day body weight.

9. The method of claim 1, wherein the solution is administered by injection to a human, at a dosage from about 0.25 ml/kg/day body weight to about 4 ml/kg/day body weight.

10. A method for treating or preventing sepsis in a animal, comprising administering an electrolyzed saline solution to the animal, wherein the electrolyzed saline solution comprises from about 0.1 ppm to about 100 ppm ozone and one or more active species selected from the group consisting of: about 5 ppm to about 300 ppm of at least one active chlorine species, about 0.1 ppm to about 300 ppm of at least one active oxygen species, about 5 ppm to about 300 ppm of at least one active hydrogen species, and combinations thereof.

11. The method of claim 2, wherein the active chlorine species comprises at least one of an active chlorine species selected from the group consisting of: free chlorine, hypochlorous acid and hypochlorite ion.

12. The method of claim 1, wherein the solution is prepared by subjecting a 1% or less saline solution to electrolysis under conditions sufficient to produce the desired active ingredients.

13. The method of claim 4, wherein the solution is prepared using a saline solution with a starting sodium chloride solution selected from the group consisting of: 0.9% NaCl (w/vol), 0.45% NaCl (w/vol), and 0.215% NaCl (wt/vol).

14. The method of claim 1, wherein the solution comprises HOCl⁻¹, OCL⁻¹, Cl⁻¹, Cl₂, O₂³, O₃, and H₂O₂.

15. The method of claim 1, wherein the solution is administered by intravenous or intraperitoneal injection.

16. The method of claim 1, wherein the solution is administered by injection to a food animal at a dosage from about 0.25 ml/kg/day body weight to about 4 ml/kg/day body weight.

17. The method of claim 1, wherein the solution is administered by injection to a human, at a dosage about 0.25 ml/kg/day body weight to about 4 ml/kg/day body weight.

18. A method for treating or preventing sepsis in a animal, comprising administering an electrolyzed saline solution to the animal, wherein the electrolyzed saline solution comprises less than about 0.8 ppm ozone and one or more active species selected from the group consisting of about 5 ppm to about 300 ppm of at least one active chlorine species, about 0.1 ppm to about 300 ppm of at least one active oxygen species, about 5 ppm to about 300 ppm of at least one active hydrogen species, and combinations thereof.

19. The method of claim 18, wherein the electrolyzed saline solution comprises about 55 ppm to about 80 ppm of at least one active chlorine species.

20. The method of claim 19, wherein the electrolyzed saline solution further comprises less than about 15 ppm hydrogen peroxide.
